(19) United States
(12) Patent Application Publication
(10) Pub. No.: US 2022/0095949 A1
(43) Pub. Date: Mar. 31, 2022

(54) NON-INVASIVE METHOD FOR DIAGNOSING CHRONIC LIVER DISEASE AND PRIMARY AND SECONDARY LIVER CANCERS

(71) Applicant: THE CLEVELAND CLINIC FOUNDATION, Cleveland, OH (US)

(72) Inventors: Daniela S. Allende, Cleveland, OH (US); Federico N. Aucejo, Cleveland, OH (US); Daniel M. Rotroff, Cleveland, OH (US)

(21) Appl. No.: 17/429,078

(22) PCT Filed: Feb. 6, 2020
(86) PCT No.: PCT/US2020/016921

§ 371 Date: Aug. 6, 2021

Related U.S. Application Data
(60) Provisional application No. 62/801,765, filed on Feb. 6, 2019.

(51) Int. Cl. A61B 5/08; A61B 5/00; G01N 33/00; G01N 33/497

(52) U.S. Cl. CPC A61B 5/082; A61B 5/4244; G01N 2033/4975; G01N 33/497; G01N 33/0047

(57) ABSTRACT

A method for diagnosing a subject with one or more of hepatocellular carcinoma (HCC), chronic liver disease, colorectal liver metastases (CRLM), and pulmonary hypertension is described. The method includes obtaining a breath sample from a subject, analyzing the breath sample obtained from the subject to determine one or more breath metabolite abundance values, inputting one or more of the breath metabolite abundance values into a machine-learning-model, and assigning a clinical parameter to the subject representing the likelihood that the subject has one or more of HCC, chronic liver disease, CRLM, and pulmonary hypertension.

### RELATED APPLICATIONS

This application claims the benefit of U.S. Provisional Application Ser. No. 62/801,765, filed Feb. 6, 2019, entitled “Non-Invasive Method for Diagnosing Pulmonary Hypertension, Chronic Liver Disease, and Primary and Secondary Liver Cancers.” This provisional application is hereby incorporated by reference in its entirety for all purposes.

### TECHNICAL FIELD

The present disclosure relates generally to methods for diagnosing chronic liver disease, primary and secondary liver cancers, and pulmonary hypertension. More specifically, the present invention relates to methods for diagnosing hepatocellular carcinoma (HCC), chronic liver disease, colorectal liver metastases (CRLM), and pulmonary hypertension based on the abundance of one or more metabolites present in the subject's breath.

### BACKGROUND

Liver cancer is a leading cause of cancer mortality, with major health implications both in the United States and globally. Hepatocellular carcinoma (HCC) accounts for 80% of liver cancers, and has been increasing due to nonalcoholic fatty liver disease and steatohepatitis (NAFLD/NASH), hepatitis C, and excessive alcohol consumption. Patients often live for years with chronic and progressive liver diseases, such as NAFLD, NASH and cirrhosis, prior to the development of HCC. Although the prevalence of NAFLD and NASH are difficult to estimate because they are often silent diseases with few or no symptoms, it is thought that up to 24% of the global population has one of these diseases due to increases in obesity, diabetes, and other metabolic disorders. This epidemic will lead to a high percentage of patients with cirrhosis who will ultimately succumb to liver failure and/or liver cancer. Furthermore, in the United States, colorectal cancer is also a leading cause of cancer deaths. Although the liver is a common site of metastasis, only 20-30% of patients with colorectal liver metastases (CRLM) are candidates for resection due to extrahepatic disease and other complicating factors. Therefore, it is critical that non-invasive, accurate, and cost-effective tools are developed that can diagnose these diseases and track their progression. The more accessible these tools are, the more they can be used to monitor development of disease for early detection and treatment.

There are several biomarkers currently used in clinical practice to diagnose primary and secondary liver cancers, however, these biomarkers suffer from poor sensitivity. These biomarkers rely on molecules secreted from tumors in order to be detected using a blood test. Unfortunately, tumors are heterogeneous and do not always secrete these molecules, resulting in many false negative diagnoses. Alpha fetoprotein (AFP) is considered the “gold standard” for detecting HCC, but is not secreted in ˜50% of HCCs, and thus, it only has a sensitivity ranging from 40-64%. Carcinoembryonic antigen (CEA) is a marker for colorectal cancer, although it also suffers from poor predictive ability with a sensitivity of approximately 50%. The liver is a common site of metastasis for patients with colorectal cancer, and it is critical that patients with CRLM are detected as early as possible in order to maximize curative treatment options.

Advanced liver disease can result in pulmonary hypertension. Pulmonary hypertension is a rare lung disorder, and patients are normally severely affected, with a life expectancy of only a few years after the first symptoms occur. Pulmonary hypertension is a chronic, progressive disease characterized by elevated blood pressure in the pulmonary arteries. Pulmonary hypertension is often asymptomatic in the beginning and is typically diagnosed late in its course. Despite improvements in the diagnosis and management of pulmonary hypertension with the introduction of targeted medical therapies leading to improved survival, the disease continues to have a poor long-term prognosis.

Hence, there is an urgent need for development of efficient diagnostic tools, particularly those enabling reliable detection of HCC, chronic liver disease, CRLM, and pulmonary hypertension at their early stages, preferably using a non-invasive approach.

### SUMMARY

In one aspect, the present disclosure provides a method of diagnosing a subject with one or more of HCC, chronic liver disease, CRLM, and pulmonary hypertension where the method comprises obtaining a breath sample from a subject, analyzing the breath sample obtained from the subject to determine one or more breath metabolite abundance values, inputting one or more of the breath metabolite abundance values into a machine-learning-model, and assigning a clinical parameter to the subject representing the likelihood that the subject has one or more of HCC, chronic liver disease, CRLM and pulmonary hypertension.

In another aspect, the present disclosure provides a method for treating a subject, the method comprising obtaining a breath sample from a subject, analyzing the breath sample obtained from the subject to determine an abundance value of one or more breath metabolites, inputting one or more of the breath metabolite abundance values into a machine-learning-model, assigning a clinical parameter to the subject representing the likelihood that the subject has one or more of HCC, chronic liver disease, CRLM, and pulmonary hypertension; and administering a treatment to the subject based on the clinical parameter.

### BRIEF DESCRIPTION OF THE FIGURES

The foregoing and other features of the present disclosure will become apparent to those skilled in the art to which the present disclosure relates upon reading the following description with reference to the accompanying drawings, in which:

FIG. 1 illustrates a functional block diagram of an exemplary system configured to predict clinical parameters related to one or more of HCC, chronic liver disease, CRLM, and pulmonary hypertension, based on breath metabolite data;

FIG. 2 illustrates a functional block diagram of a second exemplary system configured to predict clinical parameters related to one or more of HCC, chronic liver disease, CRLM, and pulmonary hypertension, based on breath metabolite data;

FIG. 3 illustrates a functional block diagram of a method for diagnosing a subject with one or more of HCC, chronic liver disease, CRLM, and pulmonary hypertension;

FIG. 4 illustrates a functional block diagram of a method for treating a subject in accordance with an aspect of the present invention;

FIG. 5 provides bar graphs of model performance metrics across disease categories in accordance with an aspect of the present invention; and

FIG. 6 provides a bar graph of the overall model balanced accuracy using patient variable and breath metabolites in accordance with an aspect of the present invention.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

Unless otherwise indicated, all numbers expressing quantities used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

Also herein, where a range of numerical values is provided, it is understood that each intervening value is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, the phrase “one or more of hepatocellular carcinoma (HCC), chronic liver disease, colorectal liver metastases (CRLM), and pulmonary hypertension” can refer to any one of the listed diseases as well as any and all combinations of the listed diseases.

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals include primates, such as simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs).

As used herein, the term “biological sample” can refer to any biological sample from a subject where the sample is suitable for metabolite analysis. Suitable biological samples for determining metabolite abundance values in a subject include but are not limited to bodily fluids such as blood-related samples (e.g., whole blood, serum, plasma, and other blood-derived samples), urine, sputum, cerebral spinal fluid, bronchoalveolar lavage, and the like. Another example of a biological sample is an exhaled breath sample. A biological sample may be fresh or stored. Biological samples may be or have been stored or banked under suitable tissue storage conditions. Biological samples can be chilled after collection in order to prevent deterioration of the sample.

As used herein, the term “metabolite” can refer to a substance such as a small molecule compound produced by a subject or patient's metabolism, or a substance that takes part in a particular metabolic process. The term metabolite, as used herein, may refer to, for example, breath metabolites. Breath metabolites can include volatile organic compounds (VOCs) of the exhaled breath of a subject.

As used herein, the term “phenotype” can refer to the physical appearance or biochemical characteristic of a subject or patient as a result of the interaction of its genotype and the environment.

As used herein, the term “diagnosis” can encompass determining the existence or nature of disease in a subject. As understood by those skilled in the art, a diagnosis does not indicate that it is certain that a subject certainly has the disease, but rather that it is very likely that the subject has the disease or the risk or probability of having the disease. A diagnosis can be provided with varying levels of certainty, such as indicating that the presence of the disease is 60% likely, 70% likely, 80% likely, 90% likely, 95% likely, or 98% likely, for example. The term diagnosis, as used herein also encompasses determining the severity and probable outcome of disease or episode of disease or prospect of recovery, which is generally referred to as prognosis.

As used herein, the term “healthy” can refer to a subject or patient that has not been identified as having any of the following: HCC, chronic liver disease (e.g., cirrhosis), CRLM, or pulmonary hypertension.

As used herein, the terms “treatment,” “treating,” and the like, can refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

As used herein, the term “abundance value” can refer to the relative or quantitative amount of a compound, such as a breath metabolite. For example, an abundance value can be a quantitative concentration value.

### II. Overview

The present disclosure relates generally to a method for diagnosing a subject with one or more of HCC, chronic liver disease, CRLM, and pulmonary hypertension based on the abundance of one or more metabolites present in the subject's breath. From this diagnosis, methods of treatment are also provided.

The present disclosure is based, at least in part, on the surprising finding that there are quantifiable differences in metabolites in the breath of patients with and without HCC, chronic liver disease, CRLM, and pulmonary hypertension that allow for identification of HCC, chronic liver disease, CRLM, and pulmonary hypertension using breath analysis. The breath metabolites that may be used to differentiate between subjects with and without HCC, chronic liver disease, CRLM, and pulmonary hypertension can include 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine among others. One or more breath metabolite abundance values can be input into a machine learning model, and the output of the machine learning model can be used to diagnose a subject with one or more of HCC, chronic liver disease, CRLM, and pulmonary hypertension. This method of diagnosing HCC, chronic liver disease, CRLM, and pulmonary hypertension allows for medical professionals to carry out rapid, point-of-care tests using a non-invasive, accurate, and cost-effective method.

### III. Methods

In one aspect, the present disclosure provides a method of diagnosing a subject with one or more of HCC, chronic liver disease, CRLM, and pulmonary hypertension where the method comprises obtaining a breath sample from a subject, analyzing the breath sample obtained from the subject to determine one or more breath metabolite abundance values, inputting one or more of the breath metabolite abundance values into a machine-learning-model, and assigning a clinical parameter to the subject representing the likelihood that the subject has one or more of HCC, chronic liver disease, CRLM and pulmonary hypertension.

In one aspect, the present disclosure provides a method of diagnosing a subject with one or more of HCC, chronic liver disease, CRLM, and pulmonary hypertension where the method first includes obtaining a breath sample from the subject using any known breath collection device.

In one instance, a breath sample may be collected using a device that includes a container. The container can be, for example, a Mylar® balloon bag, a cartridge, or a vial. In certain instances, the Mylar® bag can be re-used after flushing it with nitrogen.

In certain instances, the ambient air that is inhaled prior to collection of a subsequent breath sample can be optionally filtered. The filter can be used to prevent viral and bacterial exposure to the subject and to eliminate exogenous VOCs from the inhaled air. For example, the inhaled ambient air can be optionally filtered through an N7500-2 acid gas cartridge.

In further instances, the breath collection device can include one or more sensors. Exemplary sensors include CO₂, pressure, and temperature sensors.

In one instance, a breath sample can be collected using a device that includes a mask and one or more collection cartridges or vials. For example, a breath collection device such as that described in U.S. Patent Publication No. 2017/0303823 can be used.

In another example, a breath sample can be collected from a subject using a collection device that includes a mouthpiece, one or more filters, and a collection container. The breath sample can be collected using the following process: (i) the subject can carry out a tidal volume exhalation to clear residual air from the anatomic dead space; (ii) the subject can take a deep breath through a disposable micro filtered mouthpiece which can prevent exposure to viral and bacterial pathogens in the ambient air and eliminate exogenous VOCs; and (iii) the subject can carry out tidal volume exhalation back through the mouthpiece. The exhaled breath can be collected in a container, e.g., a Mylar balloon bag.

In another aspect, the method can further include a step in which the subject rinses their mouth with tap water immediately before the breath sample is obtained in order to eliminate contamination from oral VOCs.

In a further aspect, the breath sample may be stored or banked under suitable storage conditions.

In certain instances the breath sample can be analyzed within, for example, about 72 hours, about 24 hours, about 8 hours, about 4 hours, and about 2 hours following collection. In other instances the breath sample can be analyzed within, for example, three months, one month, or one week following collection. In yet a another example, a breath sample can analyzed within 2 hours of collection after incubation to 37° C. for 10 minutes using a Selective Ion Flow Tube Mass Spectrometer (SIFT-MS).

Once a breath sample has been obtained, an analytic device can be used to analyze the breath sample to determine the abundance of one or more breath metabolites. In certain instances, the analytic device can be a part of the breath collection device.

With recent advances in technology, it is possible to identify thousands of substances in the breath, such as breath metabolites, volatile compounds, e.g., VOCs, and elemental gases. A number of methods and analytic devices known in the art can be used to detect the presence and/or abundance of breath metabolites in a biological sample. Exemplary methods include gas chromatography (GC); spectrometry, for example mass spectrometry, and colorimetry.

A number of different forms of mass spectrometry can be used including selected-ion flow-tube mass spectrometry (SIFT-MS), thermal desorption, quadrapole, time of flight, tandem mass spectrometry, ion cyclotron resonance, and/or sector (magnetic and/or electrostatic) mass spectrometry. For example, SIFT-MS can identify trace gases in the human breath in the parts per billion, and even the parts per trillion range.

In SIFT-MS, a mixture of reagent ions (H₃O⁺, NO⁺, and O₂⁺) are generated in a microwave discharge. Each of these reagent ions can be selected by a quadrupole mass filter and separately injected into a carrier gas in a flow tube. The chosen reagent ions then react with the trace components in the sample to generate product ions. The reagent ions and product ions are mass analyzed by a quadrupole mass spectrometer and counted by a detector. The concentrations of individual compounds can be derived largely using the count rates of the precursor and product ions, and the reaction rate coefficients.

Other spectrometry methods that may be used include field asymmetric ion mobility spectrometry (FAIMS) and differential mobility spectrometry (DMS). Both DMS and FAIMS have several features that make them excellent platforms for metabolite and VOC analysis. DMS is quantitative, selective, and sensitive, with a volatile detection limit in the parts-per-trillion range. FAIMS has a volatile detection limit in the parts per billion, and in some cases parts per trillion range. The FAIMS chip can be incorporated into portable instruments making it useful for point of care operation.

In certain instances the analytic device can include one or more additional instruments, such as a separation device, that can be used to physically separate the metabolites prior to analysis. For example, the analytic device may include a high performance liquid chromatography instrument with an on-line electrospray ionization tandem mass spectrometry instrument.

The analytic device can be a portable or a stationary device.

In some aspects, the analytic device includes a gas collection component for receiving a breath sample. For example, the analytic device can be a mass spectrometry device with a Mylar collection bag attached directly to it.

The analytic device can be used to identify one or more breath metabolites and determine the abundance of the one or more metabolites in the sample. In one example, the breath metabolites can include one or more of the following: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methyl hexane, (E)-2-nonene, ammonia, ethane, hydrogen sulfide, triethylamine, and trimethylamine. One skilled in the art would understand that an analytic device may be able to detect many other metabolites in a subject's breath.

Following breath analysis, one or more of the breath metabolite abundance values can be input into a machine learning model. The machine learning model can diagnose a subject with one or more of HCC, chronic liver disease, CRLM and pulmonary hypertension. In one instance, the machine learning model can diagnose a subject with HCC. In another instance, the machine learning model can diagnose a subject with chronic liver disease. In a further instance, the machine learning model can diagnose a subject with CRLM. In yet another instance, the machine learning model can diagnose a subject with pulmonary hypertension. In another instance, the machine learning model can diagnose a subject as having, for example, two conditions selected from HCC, chronic liver disease, CRLM and pulmonary hypertension (e.g., pulmonary hypertension and chronic liver disease). More specifically, the machine learning model can provide the likelihood that the subject has one or more of HCC, chronic liver disease, CRLM or pulmonary hypertension. In certain instances, a diagnosis of HCC, chronic liver disease, CRLM or pulmonary hypertension can indicate that the subject is at least 60%, 70%, 80%, or 90% likely to have the indicated condition.

A number of machine learning models can be generated to predict whether or not a subject has HCC, chronic liver disease, CRLM or pulmonary hypertension.

Machine Learning

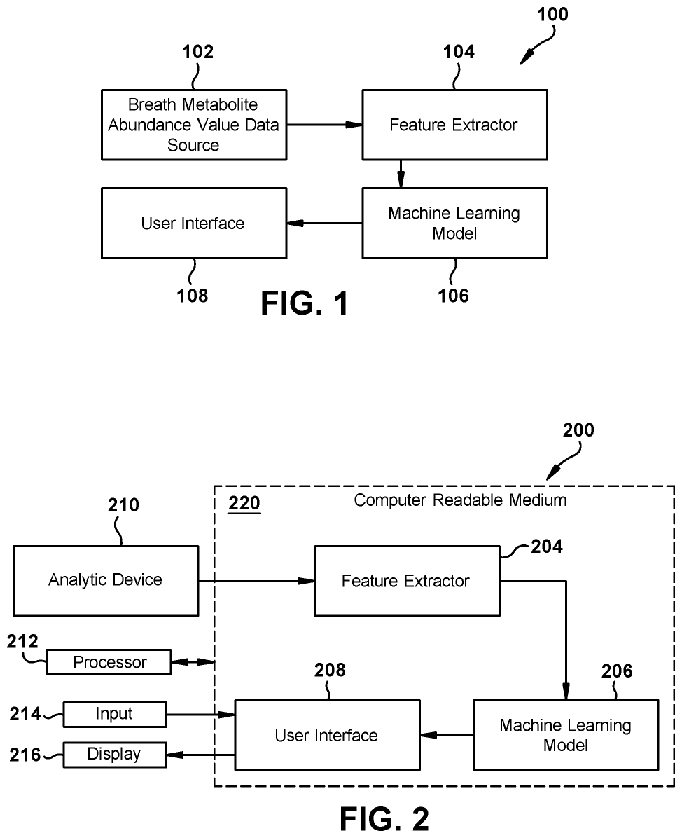

One aspect of the present disclosure is shown in FIG. 1. FIG. 1 illustrates a functional block diagram of an example of a system 100 for predicting whether or not a subject has one or more of HCC, chronic liver disease, CRLM and pulmonary hypertension based on the subject's breath metabolite abundance values. The system 100 can be implemented on one or more physical devices (e.g., servers) that may reside in a cloud computing environment or on a computer, such as a laptop computer, a desktop computer, a tablet computer, a workstation, or the like. In the present example, although the components 102, 104, 106, and 108 of the system 100 are illustrated as being implemented on the same system, in other examples, the different components could be distributed across different systems and communicate, for example, over a network, including a wireless network, a wired network, or a combination thereof. The system 100 includes a breath metabolite abundance value data source 102 that can be accessed to provide one or more breath metabolite abundance values. The breath metabolite abundance value data source 102 can include, for example, the analytic device used to identify and determine the abundance of one or more breath metabolites. The breath metabolite abundance data source may also contain a storage medium accessible by a local bus or a network connection, or a user interface at which a user can enter information from a previously obtained breath metabolite analysis profile.

A feature extractor 104 can generate a feature vector representing the subject from the breath metabolite abundance values. The abundance values can be relative or quantitative abundance values. For example, the feature extractor 104 can utilize the absolute or normalized quantity or concentration of one or more of the breath metabolites or one or more values derived from the breath metabolite quantities. In one aspect, a metabolite abundance value is relative to the other metabolite abundance values input into the machine-learning model. It will be appreciated that the feature extractor 104 can also utilize additional parameters, for example, general patient variables of the subject such as age, sex, and basil metabolic index (BMI), and other medical diagnoses. In some instances, the feature extractor 104 can use age, sex, and BMI. In further instances, the feature extractor 104 can use age and sex. These parameters can be provided, for example, from an electronic health records database via a network interface (not shown) or via a user interface 106. A machine learning model 106 determines at least one clinical parameter for the subject from the metric. It will be appreciated that the clinical parameter can represent, for example, the probability that the subject has HCC, chronic liver disease, CRLM or pulmonary hypertension or the probability that the subject will respond to treatment for HCC, chronic liver disease, CRLM or pulmonary hypertension. The clinical parameter provided by the machine learning model 106 can be stored on a non-transitory computer readable medium associated with the system and/or provided to a user at a display via the user interface 108.

FIG. 2 illustrates a functional block diagram of an example of a system 200 for predicting clinical parameters related to HCC, chronic liver disease, CRLM and pulmonary hypertension. To this end, the system 200 incorporates a machine learning model 206 that generates a clinical parameter representing, for example, a HCC, chronic liver disease, CRLM or pulmonary hypertension diagnosis or the probability that a subject will respond to treatment for HCC, chronic liver disease, CRLM or pulmonary hypertension. In the illustrated implementation, an analytic device 210 provides breath metabolite abundance value data, for example, the relative or quantitative amount of one or more breath metabolites detected, to a data analysis component implemented as a general purpose processor 212 operatively connected to a non-transitory computer readable medium 220 storing machine executable instructions. An input device 214, such as a mouse or a keyboard, is provided to allow a user to interact with the system, and a display 216 is provided to display breath metabolite abundance data and calculated parameters to the user. For example, the display 216 can display a preliminary diagnosis or a likely diagnosis.

The machine learning model 206 can utilize one or more pattern recognition algorithms, implemented, for example, as classification and regression models, each of which analyze the extracted feature vector to assign a clinical parameter to the user. It will be appreciated that the clinical parameter can be categorical or continuous. For example, a categorical parameter can represent the presence or absence of HCC, chronic liver disease, CRLM or pulmonary hypertension, expected efficacy of the treatment, or binned ranges of likelihood of these categories. A continuous parameter can represent, for example, a likelihood that the subject has HCC, chronic liver disease, CRLM or pulmonary hypertension or a likelihood that the subject will respond to treatment.

Where multiple classification and regression models are used, the machine learning model 206 can include an arbitration element that can be utilized to provide a coherent result from the various algorithms. Depending on the outputs of the various models, the arbitration element can simply select a class from a model having a highest confidence, select a plurality of classes from all models meeting a threshold confidence, select a class via a voting process among the models, or assign a numerical parameter based on the outputs of the multiple models. Alternatively, the arbitration element can itself be implemented as a classification model that receives the outputs of the other models as features and generates one or more output classes for the patient.

The classification can also be performed across multiple stages. In one example, the patient variables or clinical parameters for the subject can be used with a first stage of the machine learning model to generate an a priori probability that the subject has HCC, chronic liver disease, CRLM or pulmonary hypertension. The breath metabolite abundance values for the subject can then be determined and used at a second stage of the machine learning model to generate a classification for the subject as having HCC, chronic liver disease, CRLM or pulmonary hypertension or not having HCC, chronic liver disease, CRLM or pulmonary hypertension. A known performance of the second stage of the machine learning model, for example, defined as values for the specificity and sensitivity of the model, can be used to update the a priori probability given the output of the second stage.

The machine learning model 206, as well as any constituent models, can be trained on training data representing the various classes of interest. The training process of the machine learning model 206 will vary with its implementation, but training generally involves a statistical aggregation of training data into one or more parameters associated with the output classes. Any of a variety of techniques can be utilized for the models, including support vector machines (SVM), regression models, self-organized maps, k-nearest neighbor (KNN) classification or regression, fuzzy logic systems, data fusion processes, boosting and bagging methods, rule-based systems, or artificial neural networks (ANN).

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A k-nearest neighbor model populates a feature space with labelled training samples, represented as feature vectors in the feature space. In a classifier model, the training samples are labelled with their associated class, and in a regression model, the training samples are labelled with a value for the dependent variable in the regression. When a new feature vector is provided, a distance metric between the new feature vector and at least a subset of the feature vectors representing the labelled training samples is generated. The labelled training samples are then ranked according to the distance of their feature vectors from the new feature vector, and a number, k, of training samples having the smallest distance from the new feature vector are selected as the nearest neighbors to the new feature vector.

In the classifier model, the class represented by the most labelled training samples in the k nearest neighbors is selected as the class for the new feature vector. In a regression model, the dependent variable for the new feature vector can be assigned as the average of the dependent variables for the k nearest neighbors. It will be appreciated that k is a metaparameter of the model that is selected according to the specific implementation. The distance metric used to select the nearest neighbors can include a Euclidean distance, a Manhattan distance, or a Mahalanobis distance.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector.

A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used, but a continuous parameter can be computed according to a number of decision trees that select a given task.

A Random Forest model can be optimized by investigating the classification accuracy on a set of training and test data to determine the optimal number of decision trees (ntrees) that should be utilized to construct the forest and to determine the optimal number of randomly selected features (mtry) that should be made available to be utilized at each node of the tree. Cross-validation can be used to evaluate the performance of the model which entails iteratively removing an individual subject or a set of subjects and using the data for model training. The withheld subject or set of subjects can then used to evaluate the performance of the model. This process is repeated until all subjects have been used in both model training and model testing. The numbers of trees (ntrees) can vary and can range from about 50 to about 1000. In one example, the number of trees is 50. In a further example, the number of trees can be at least about 50, 100, 150, 200, 250, 300, 350, 400, 450, 500, 550, 600, 650, 700, or 750 trees. The number of randomly selected features, mtry, can vary, for example, from 1-40 features. In one example, the number of randomly selected features can be from 1-24 features. In one aspect of the present invention the optimal set of parameters can be selected based on a maximization of the classification accuracy in the withheld subjects (test set).

In another aspect of the present invention, a classification model can include a leave-one-out cross validation (LOOCV) approach that can be used during all parameter combinations. For example, a method herein can use n−1 subjects during model training followed by testing on the withheld subject. The entire process can be repeated n-times until each sample is used as a test case and the mean accuracy can be calculated as an indicator of model performance. The parameters that result in the model with the highest mean classification accuracy can be used to develop the final model.

Accuracy, sensitivity, specificity, and balanced accuracy can be measured for the classification model(s) that is used. Sensitivity measures the proportion of true positives out of the number of identified positives. Specificity, or true negative rate, is the number of true negatives divided by the number of true negatives plus false positives. Specificity measures the proportion of true negatives out of all the negatives identified. Balanced accuracy is the mean of sensitivity and specificity. Accordingly, the sensitivity of the classification models according to the present disclosure include the models of a) breath metabolites abundance values only, b) patient variables (for example age, body mass index (BMI), and sex of subject) only, and c) metabolites and clinical variables, have at least one of a sensitivity, specificity, accuracy and balanced accuracy of at least about 50%, in another example at least about 55%, in another example at least about 60%, in another example at least about 65%, in another example at least about 70%, in another example at least about 75%, in another example at least about 80%, in another example at least about 85%, in another example at least about 85%, in another example at least about 90%, in another example at least about 95% and in another example at least about 97% and in another example at least about 98%.

Regardless of the specific model employed, the clinical parameter generated at the machine learning model 206 can be provided to a user at the display 216 via a user interface 208 or stored on the non-transitory computer readable medium 220, for example, in an electronic medical record associated with the patient.

In one aspect, the machine learning model is generated using breath metabolite abundance values of subjects where the subject's HCC, chronic liver disease, CRLM or pulmonary hypertension diagnosis is already known.

In another aspect, the machine learning model is generated using a Random Forest classification model. In one instance, the breath metabolite abundance values that can be used to generate the machine learning model include abundance values for one or more of the following: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methyl hexane, (E)-2-nonene, ammonia, ethane, hydrogen sulfide, triethylamine, and trimethylamine. In some instances, the abundance values of all of the aforementioned breath metabolites are used to generate the machine learning model. In further instances, the abundance values used to generate the machine learning model include ethane, acetaldehyde, (E)-2-nonene, and acetone abundance values. In other instances, patient variables such as age, sex, and BMI can also be used to generate the machine learning model.

Provided herein are methods for diagnosing a subject with one or more of hepatocellular carcinoma (HCC), chronic liver disease, colorectal liver metastases (CRLM), and pulmonary hypertension. In one aspect, method 300 includes obtaining a breath sample from a subject 302, analyzing the breath sample obtained from the subject to determine one or more breath metabolite abundance values 304, inputting one or more of the breath metabolite abundance values into a machine-learning-model 306, and assigning a clinical parameter to the subject representing the likelihood that the subject has one or more of HCC, chronic liver disease, CRLM and pulmonary hypertension 308. In certain aspects, the one or more breath metabolites are selected from the following: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine.

In certain aspects, the method includes inputting one or more of 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine abundance values into the machine-learning-model. In certain aspects, the method includes inputting 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine abundance values into the machine-learning-model. In other aspects, the method includes inputting ethane, acetaldehyde, (E)-2-nonene, and acetone abundance values into the machine-learning-model. In further instances, the method can include inputting age, sex, and BMI patent variables into the machine-learning-model. In even further instances, the method can include inputting age and sex patent variables into the machine-learning-model.

Also provided herein are methods for treating a subject. In one aspect, the method 400 includes obtaining a breath sample from a subject 402, analyzing the breath sample obtained from the subject to determine one or more breath metabolite abundance values 404, inputting one or more of the breath metabolite abundance values into a machine-learning-model 406, assigning a clinical parameter to the subject representing the likelihood that the subject has one or more of HCC, chronic liver disease, CRLM, and pulmonary hypertension 408, and administering a treatment to the subject based on the clinical parameter 410. If the subject is diagnosed with one or more of HCC, chronic liver disease, CRLM, or pulmonary hypertension, the subject can be treated accordingly.

Treatment for HCC and/or CRLM can include surgery, liver transplant surgery, ablation procedures, chemotherapy, radiation therapy, and immunotherapy. Treatment for chronic liver disease can comprise treatment for alcohol dependency, weight loss, medications including medications to treat hepatitis, and liver transplant surgery. Treatment for pulmonary hypertension can include administering certain medications such as vasodilators, endothelin receptor antagonists, sildenafil and tadalafil, calcium channel blockers, soluble guanylate cyclase stimulators, anticoagulants, digoxin, diuretics, and oxygen. Treatment for pulmonary hypertension can also include surgery or a lung or heart transplant.

In some embodiments, the methods described herein include performing an additional diagnostic test for HCC, chronic liver disease, CRLM, or pulmonary hypertension. A number of such tests are known in the art and include blood tests, imaging tests, and biopsies.

### IV. Experimental

The following example is for the purpose of illustration only is not intended to limit the scope of the appended claims.

Exhaled Breath Collection

SIFT-MS breath analysis was performed on all subjects to measure breath metabolites, including VOCs, in the exhaled breath. The age, gender, and BMI were recorded for each subject.

All subjects completed a mouth rinse with water prior to the collection of the breath sample in order to reduce the contamination from VOCs produced in the mouth. Subjects were prompted to exhale normally to release residual air from the lungs and then inhale to lung capacity through a disposable mouth filter. The inhaled ambient air was also filtered through an attached N7500-2 acid gas cartridge. The filters were used to prevent viral and bacterial exposure to the subject and to eliminate exogenous VOCs from the inhaled air. The exhaled breath sample was collected into an attached Mylar® bag, capped, and analyzed within four hours. Mylar® bags were cleaned by flushing with nitrogen between subjects.

The concentration of 22 metabolites known in exhaled breath were measured. The measured compounds included: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine and trimethylamine.

The distributions for each metabolite concentration were log-transformed to normalize the data. At this point the two data sets, clinical variables and metabolite values, were combined.

Metabolite Data Processing

Histograms were plotted for each variable in order to assess whether data transformations were necessary. Most of the variables demonstrated some right skew and long tails. All clinical variables were log transformed to conform to assumptions of normality. Principal Components Analysis (PCA) was conducted on the clinical variables in order to detect any potential batch effects or outlying samples. Two outlying samples were removed from further analyses. The distributions for each metabolite concentration were log-transformed to normalize the data.

Random Forest Ensemble Classification

A random forest ensemble classification approach was implemented to determine if combinations of known metabolites and patient variables could accurately classify patients by disease status. Models were developed that included i) metabolites only, ii) patient variables (i.e., age, BMI, sex) only, and iii) metabolites and patient variables. Random forest was implemented using the R package, Random Forest (Liaw and Wiener 2002).

A grid search was performed to optimize the hyperparameters used by the random forest model. The optimal number of decision trees (ntrees) was evaluated from 100, 250, 500, 750, and 1,000 and the number of randomly selected variables selected at each node in the decision tree (mtry) was evaluated from 1 to 24 (the total number of predictors). Each unique parameter combination was tested. The grid search identified the optimal number set of parameters as 100 and 16, for ntrees and mtry, respectively.

To protect against overfitting, a leave-one-out cross validation (LOOCV) approach was used during all parameter combinations, which used n−1 subjects during model training and then tested on the withheld subject. The entire process was repeated n-times until each sample had been used as a test case and the mean accuracy was calculated as an indicator of model performance. The parameters that resulted in the model with the highest mean classification accuracy were used to develop the final model.

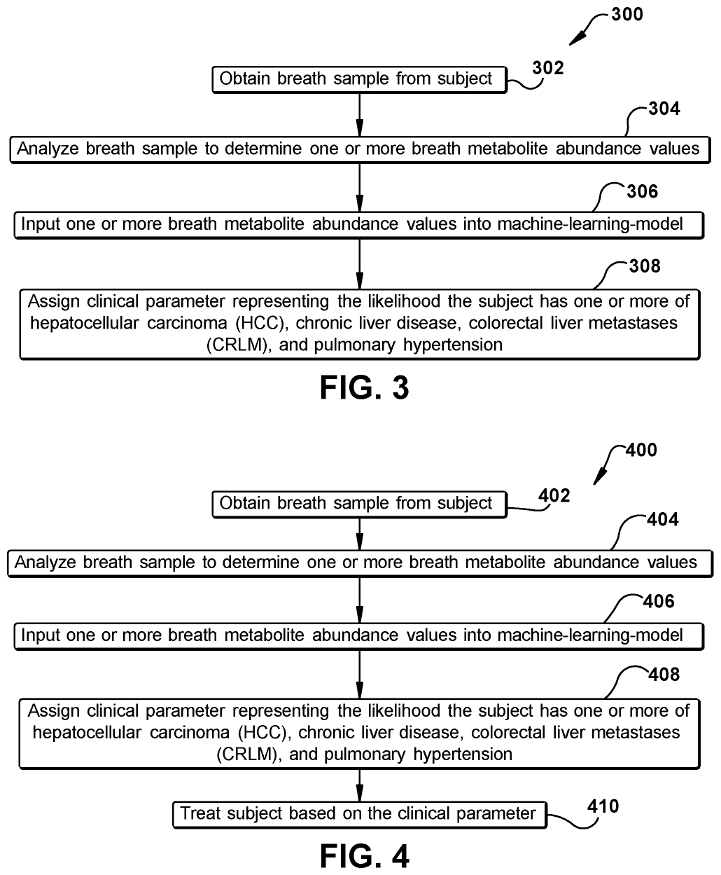

The final model was created using the optimal parameters and LOOCV. Mean classification accuracy, sensitivity, specificity, and balanced accuracy on the withheld (test) subjects were used to evaluate the model's predictive ability. FIG. 3 shows the various model performance metrics across the disease categories. Five different metrics for assessing model predictive ability are presented in FIG. 3 representing misclassification rate, accuracy, sensitivity, specificity and balanced accuracy. All metrics were generated using the withheld subject during the LOOCV (test cohort). For each disease, models using 1) only patient variables of age, sex and body mass index (BMI); 2) only breath metabolites; and 3) both patient variables and breath metabolites were developed. As used herein, classification accuracy refers to the number of correctly identified subjects (true positives and true negatives) divided by the total number of subjects.

The mean decrease Gini estimates, averaged over the n-times from the LOOCV, was used to provide an estimate of the importance of each feature to the performance of the model. Clinical data (i.e., patient variables) and metabolite mass spectrometry samples were analyzed separately and then combined for machine learning and further analysis.

Model Predictions

Once the optimal combination of parameters was found, the final model was created and LOOCV was used again to create the final accuracy estimates. Accuracy, sensitivity, specificity, and balanced accuracy were all measured with the final model. Accuracy is the number of correctly identified samples (true positives and true negatives) divided by the total number of samples. Sensitivity, or true positive rate, is the number of true positives divided by the number of true positives plus the number of false negatives. Sensitivity measures the proportion of true positives out of the number of identified positives. Specificity, or true negative rate, is the number of true negatives divided by the number of true negatives plus false positives. Specificity measures the proportion of true negatives out of all the negatives identified. Balanced accuracy is the mean of sensitivity and specificity.

Table 1 summarizes the number of patients that were evaluated and their disease classification.

TABLE 1

| Diagnosis | N |
|---|---|
| Healthy | 54 |
| Pulmonary Hypertension | 49 |
| CRLM | 51 |
| Cirrhosis | 30 |
| HCC | 112 |

Results

The optimized model's algorithm combined all 22 metabolites in a way that resulted in an average classification accuracy of 85% across the five diagnoses.

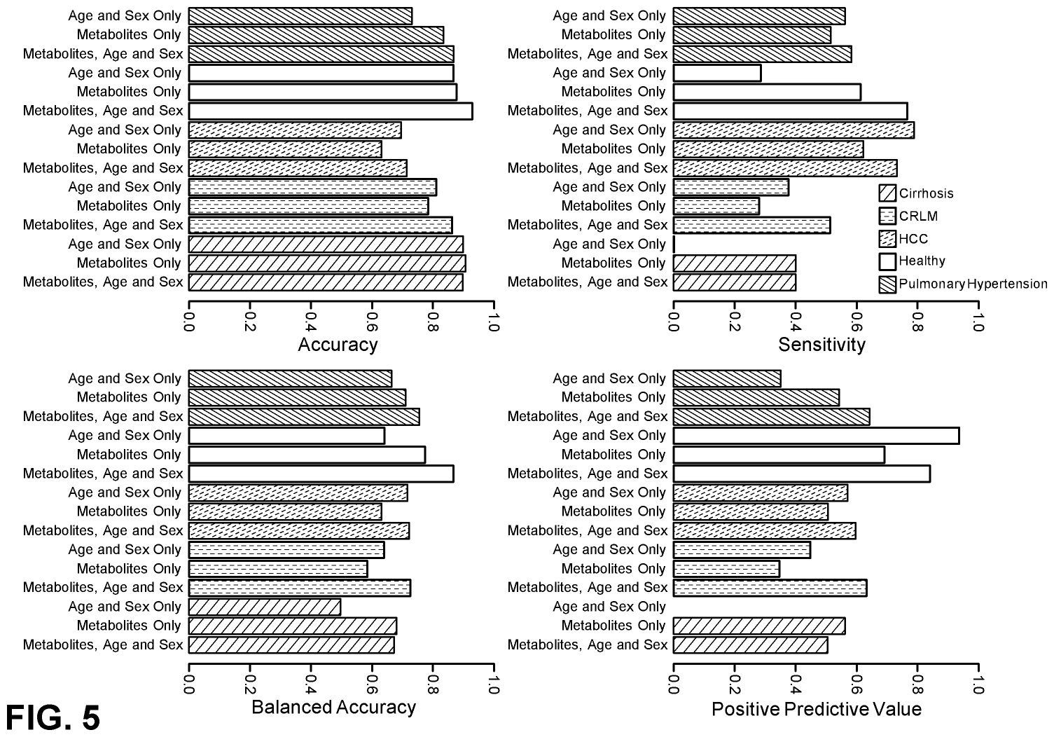

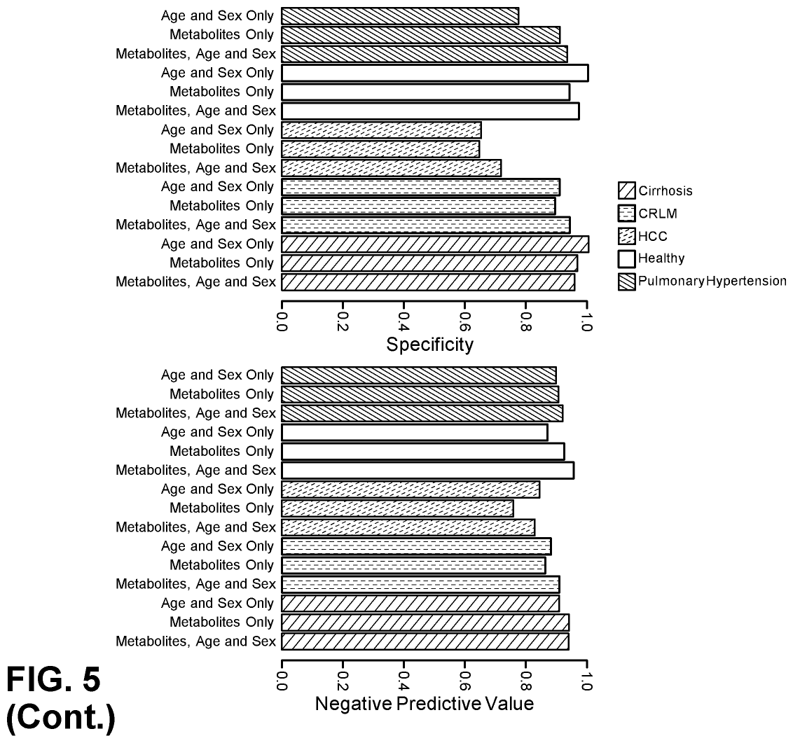

FIG. 5 illustrates bar graphs of model performance metrics across disease categories, in accordance with an example of the present disclosure.

Individual sensitivity, specificity, and balanced accuracies for each diagnosis were determined. Table 2 summarizes the metrics for the final predictive model on the withheld test subjects.

TABLE 2

| | | | Balanced |
|---|---|---|---|
| Phenotype | Sensitivity (%) | Specificity (%) | Accuracy (%) |
| Healthy | 76 | 97 | 86 |
| CRLM | 51 | 94 | 72 |
| HCC | 73 | 71 | 72 |
| Cirrhosis | 40 | 96 | 68 |
| Pulmonary | 57 | 93 | 75 |
| Hypertension | | | |

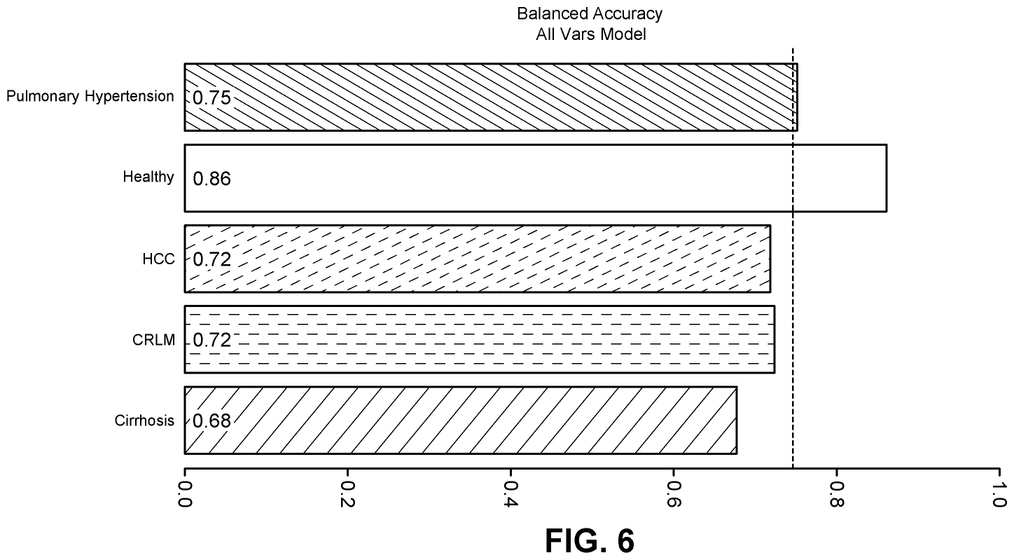

FIG. 6 provides a bar graph of the overall model balanced accuracy using patient variables and breath metabolites, in accordance with an aspect of the present invention.

Table 3 presents the results of the final predictive model parameters or features listed from most important to least important, as determined by Random Forest Gini Score.

TABLE 3

| | |
|---|---|
| Age | 19.64 |
| Ethane | 13.83 |
| Acetaldehyde | 13.10 |
| (E)-2-Nonene | 13.08 |
| Acetone | 12.43 |
| Sex | 10.43 |
| Trimethylamine | 8.48 |
| 2-Propanol | 8.39 |
| 3-Methylhexane | 7.76 |
| Acrylonitrile | 7.75 |
| Benzene | 7.59 |
| Dimethyl Sulfide | 6.52 |
| Hydrogen Sulfide | 6.39 |
| Isoprene | 6.39 |
| Ethanol | 5.66 |
| 1-Octene | 5.38 |
| Carbon Disulfide | 5.33 |
| Ammonia | 4.63 |
| 1-Nonene | 4.58 |
| Pentane | 4.52 |
| 1-Heptene | 4.48 |
| 1-Decene | 4.44 |
| Acetonitrile | 4.32 |
| Triethylamine | 4.21 |

The complete disclosure of all patents, patent applications, and publications, and electronically available material cited herein are incorporated by reference. The foregoing detailed description and examples have been given for clarity of understanding only. No unnecessary limitations are to be understood therefrom. Although the invention has been described with reference to several specific embodiments, the invention is not limited to the exact details shown and described, for variations obvious to one skilled in the art will be included. The description is not meant to be construed in a limited sense. Various modifications of the disclosed embodiments, as well as alternative embodiments of the inventions will become apparent to persons skilled in the art upon the reference to the description provided herein. It is, therefore, contemplated that the appended claims will cover such modifications that fall within the scope of the disclosure.

We claim:

1. A method of diagnosing a subject with one or more of hepatocellular carcinoma (HCC), chronic liver disease, colorectal liver metastases (CRLM), and pulmonary hypertension the method comprising:
obtaining a breath sample from a subject;
analyzing the breath sample obtained from the subject to determine one or more breath metabolite abundance values;
inputting one or more of the breath metabolite abundance values into a machine-learning-model; and
assigning a clinical parameter to the subject representing the likelihood that the subject has one or more of HCC, chronic liver disease, CRLM and pulmonary hypertension.

2. The method of claim 1, wherein the method further comprises inputting one or more patient variables into the machine-learning-model.

3. The method of claim 2, wherein the one or more patient variables are selected from the group consisting of: age, sex, and basil metabolic index (BMI).

4. The method of claim 3, wherein the patient variables are age and sex.

5. The method of claim 1, wherein the one or more breath metabolites are selected from the group consisting of: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine.

6. The method of claim 1, wherein the method comprises inputting ethane, acetaldehyde, (E)-2-nonene, and acetone abundance values into the machine-learning-model.

7. The method of claim 6, wherein the method further comprises inputting age and sex into the machine-learning-model.

8. The method of claim 1, wherein the method comprises inputting 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine abundance values into the machine-learning-model.

9. The method of claim 8, wherein the method further comprises inputting age and sex into the machine-learning-model.

10. The method of claim 1, wherein the one or more abundance values are relative abundance values.

11. The method of claim 1, wherein the one or more abundance values are quantitative concentration values.

12. The method of claim 1, wherein the machine-learning-model comprises a Random Forest classification model.

13. The method of claim 1, wherein the machine-learning-model comprises a Random Forest model classification model and a number of trees used in the Random Forest classification model is at least 50.

14. The method of claim 1, wherein the breath sample is analyzed using an analytic device.

15. The method of claim 14, wherein the analytic device is portable.

16. The method of claim 14, wherein the analytic device comprises a gas collection component for receiving the breath sample, and a sensor configured to detect the abundance of each of the one or more breath metabolites.

17. The method of claim 1, wherein the breath sample is analyzed using selective ion flow tube mass spectrometry.

18. A method for treating a subject, the method comprising
obtaining a breath sample from a subject;
analyzing the breath sample obtained from the subject to determine one or more breath metabolite abundance values;
inputting one or more of the breath metabolite abundance values into a machine-learning-model;
assigning a clinical parameter to the subject representing the likelihood that the subject has one or more of HCC, chronic liver disease, CRLM, and pulmonary hypertension; and
administering a treatment to the subject based on the clinical parameter.

19. The method of claim 18, wherein the treatment comprises surgery.

20. The method of claim 18, wherein the method comprises inputting 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethylamine, and trimethylamine abundance values into the machine-learning-model.
